(19) United States
(12) Patent Application Publication
(10) Pub. No.: US 2023/0397307 A1
(43) Pub. Date: Dec. 7, 2023

(54) LIGHTING SYSTEM

(71) Applicant: SIGNIFY HOLDING B.V., EINDHOVEN (NL)

(72) Inventors: TIES VAN BOMMEL, HORST (NL); RÉMY CYRILLE BROERSMA, EINDHOVEN (NL)

(21) Appl. No.: 18/032,589

(22) PCT Filed: Oct. 14, 2021
(86) PCT No.: PCT/EP2021/078469

§ 371 Date: Apr. 19, 2023

(30) Foreign Application Priority Data
Oct. 19, 2020 (EP) 20202489.9

(51) Int. Cl. H05B 45/20; H05B 47/16; H05B 47/115; A61L 9/22

(52) U.S. Cl. CPC H05B 45/20; H05B 47/16; H05B 47/115; A61L 9/22; A61L 2209/111; A61L 2209/12

(57) ABSTRACT

The present invention relates to a lighting system (10), comprising: a plurality of light sources (12a, 12b) adapted to emit light; a controller (14) adapted to individually control at least a first light source (12a) and a second light source (12b) of the plurality of light sources, such that at least one of color and color temperature of combined light (16) emitted by the plurality of light sources can be varied; and an air ionizer (18) adapted to generate ionized air (20), wherein the air ionizer is configured to vary its generation of ionized air as a function of at least one of the color and color temperature of the combined light emitted by the plurality of light sources.

### FIELD OF THE INVENTION

The present invention relates to a lighting system adapted to mimic natural light. The present invention also relates to a method of controlling a lighting system.

### BACKGROUND OF THE INVENTION

Illumination systems with selectively controlled illumination sources to produce conditions that mimic natural light are known, for example from US2020103841. However, it is desired to improve mimicking natural light in rooms such as offices and homes which is perceived as familiar and pleasant.

### SUMMARY OF THE INVENTION

It is an object of the present invention to provide an improved lighting system, which lighting system in particular may offer an experience which so far could only be enjoyed outside, in nature.

According to a first aspect of the invention, this and other objects are achieved by a lighting system, comprising: a plurality of light sources adapted to emit light; a controller adapted to individually control at least a first light source and a second light source of the plurality of light sources, such that at least one of color and color temperature of combined light emitted by the plurality of light sources can be varied; and an air ionizer adapted to generate ionized air, wherein the air ionizer is configured to vary its generation of ionized air either by being programmed to do so or by being controlled by a controller, in response to the controller varying at least one of the color and color temperature of the combined light emitted by the plurality of light sources, wherein said controller is either the same controller as the controller for the light sources or a different controller.

The present invention is based on the understanding that by adding an air ionizer which is configured to set its ionization depending on the present color and/or color temperature of the emitted light, a system which better mimics natural conditions (i.e. not only natural light) can be achieved. High ion concentrations are for example typical in forests where greenish white light is present. Furthermore, the present lighting system may have a specific advantage with respect to disinfection over conventional stand-alone air ionizers. Namely, as the present lighting system, and in particular several such lighting systems, typically may be mounted on or into a ceiling to function as ceiling lighting, a distributed network of air ionizers from the ceiling having better homogeneous coverage of the space/room below the ceiling (like a shower) may be realized, compared to an ionizer positioned on or near the floor.

The first light source could for example be a cool white (CW) LED and the second light source could be a warm white (WW) LED. Alternatively, the first light source could for example be a red light source of an RGB LED and the second light source could be a green light source of the RGB LED, wherein a third light source of the plurality of light sources could be a blue light source of the RGB LED.

The air ionizer may be or comprise a (negative) ion generator. That is, the air ionizer may be adapted may ionize (electrically charge) air molecules. The air ionizer may be configured to vary (increase/decrease) its generation of ionized air, i.e. its ionization, for example by being programmed to do so or by being controlled by a controller, which may be the same controller as the controller adapted to individually control at least a first light source and a second light source of the plurality of light sources or a different controller.

It should be noted that US20110128738 discloses a lighting apparatus that includes a light source and an ion generating unit. However, in US20110128738 a controller is configured to drive the plurality of ion generators such that an amount of generated ion becomes large/small in response to a turn-on/turn-off of the light source and/or high/low of illuminance thereof. Typically, according to US20110128738, the turn-on/turn-off of the light source and the high/low of the illuminance often correspond to a presence/absence of a man and a degree of activeness in human activities. Hence, US20110128738 does not disclose spectral distribution dependent ionized air generation as in the present invention.

The air ionizer may be configured to increase its generation of ionized air from a first ionized air concentration to a second ionized air concentration to produce a higher ionized air concentration when the controller controls at least the first and second light sources such that the color temperature of the combined (white) light emitted by the plurality of light sources is increased from a first color temperature, such as less than 3000K (which may correspond to warm white light), to a second color temperature, such as 3000K-4500K (which may correspond to cool white light). That is, an increase in color temperature may coincide with an increase in ionized air generation.

The second ionized air concentration may be at least 1.5 times the first ionized air concentration, wherein the second color temperature minus the first color temperature is at least 500K.

Furthermore, the air ionizer may be configured to further increase its generation of ionized air from the second ionized air concentration to a third ionized air concentration when the controller controls at least the first and second light sources such that the color temperature of the combined light emitted by the plurality of light sources is further increased from the second color temperature to a third color temperature, such as greater than 4500K (day-light). The third ionized air concentration may be at least 1.5 times the second ionized air concentration, wherein the third color temperature minus the second color temperature is at least 500K.

Preferably, the further increase in generation of ionized air when the color temperature of the combined light emitted by the plurality of light sources is further increased from the second color temperature to the third color temperature is steeper than the increase in generation of ionized air when the color temperature of the combined light emitted by the plurality of light sources is increased from the first color temperature to the second color temperature. ‘Steeper’ may here mean more generated ionized air per increased K. A boost in ionized air seems to be desired at mimicked day-light, and the steeper increase also leads to high disinfection performance.

Furthermore, at least one of the increase in generation of ionized air and the increase in color temperature, preferably both, may be gradual. This may serve to better mimic natural events/conditions, such as the transition from morning to day or day to evening. The air ionizer may (further) be configured to increase its generation of ionized air to produce a higher ionized air concentration when the controller controls at least the first and second light sources such that combined white light emitted by the plurality of light sources becomes more blueish and/or greenish to produce white light with a green or blue tint. White light with a blue tint may correspond to/mimic sky-light, and white light with a green tint may correspond/mimic to forest-light. The “combined white light” is preferably <5 SDCM (Standard Deviation Colour Matching) from the black body line (BBL). The white light with a green tint may be at least 10 SDCM from the BBL. The white light with a blue tint may be >7000K (on or near the BBL), preferably >10000K (on or near the BBL), more preferably >12000K (on or near the BBL), or at least 10 SDCM from the BBL.

Specifically, the air ionizer may be configured to further increase its generation of ionized air (e.g. from the second ionized air concentration to a higher ionized air concentration) when the controller controls at least the first and second light sources such that a color point of the combined light emitted by the plurality of light sources moves away from the second color temperature to a position resulting in that white light with a green or blue tint is produced. The higher ionized air concentration may be at least 1.5 times the second ionized air concentration.

The increase in generation of ionized air (when the combined light changes from white to blueish/greenish white) may be gradual. The air ionizer may be configured to produce a minimum ionized air concentration in the range of 25-2000 ions per cm³ and/or a maximum ionized air concentration in the range of 25000-500000 ions per cm³. In this way, the present lighting system may protect persons from the spread of bacteria and viruses such as influenza or against the outbreak of novel viruses like COVID-19. The minimum/maximum ionized air concentration may apply (homogeneously) to the whole space/room (typically after some (predetermined) settling time) in which the lighting device is installed, given that the space/room is not larger than a predefined maximum space/room volume for the air ionizer of the lighting system. Nevertheless, when the present lighting system is mounted for example in the ceiling of an office, the air ionizer may be directed to provide sufficient levels of ionized air concentration (e.g. for disinfection) is breathing areas (where people are sitting or standing; where aerosols are most present) and at desk surfaces (where droplets are most present).

The air ionizer may be configured to provide the maximum ionized air concentration (or an ionized air concentration greater than the aforementioned third ionized air concentration) when the controller controls at least the first and second light sources such that the combined light emitted by the plurality of light sources is white light with a green or blue tint. Such light may mimic a forest or waterfall, which are places where very high ionized air concentrations naturally occur.

The lighting system may be configured such that the white light with a green or blue tint is dynamically varied as a function of time. The lighting system may for example comprises means for varying at least one of the amount, position, beam shape, beam size, and pattern of (in particular green or blue light of) the emitted white light with a green or blue tint as function of time. The lighting system may for example project moving green light in white light, to really mimic moving leaves. Or the lighting system may project moving blue light in white light, to really mimic moving a waterfall. These are typically the applications where in nature there are the highest ion concentrations.

The lighting system may further comprise a presence sensor adapted to detect at least one of presence and movement of one or more persons in the vicinity of the lighting system, wherein the air ionizer is configured to vary its generation of ionized air based on input from the presence sensor. The air ionizer may for example be configured to increase its generation of ionized air, or set a high generation of ionized air, in response to the presence sensor detecting presence of one or more persons, and to decrease its generation of ionized air, or set a low generation of ionized air, in response to the presence sensor not detecting presence of any person.

The controller may further be adapted to control at least the first light source and the second light source of the plurality of light sources such that the intensity of combined light emitted by the plurality of light sources can be varied, wherein the air ionizer is configured to vary its generation of ionized air (further) as a function the intensity of any combined light emitted by the plurality of light sources. The air ionizer may for example be configured to increase (or decrease) its generation of ionized air to produce a higher (or lower) ionized air concentration when the controller controls at least the first and second light sources such that the intensity of the combined light emitted by the plurality of light sources is increased (or decreased). Varying the ionization based on both intensity and color/color temperature may provide synergistic effects: at higher color temperatures, a higher intensity is desired; and in case of blue-white or green-white, the intensity may also be higher.

Furthermore, the air ionizer may be configured to increase its generation of ionized air to produce a higher ionized air concentration when the plurality of light sources are turned off or when the controller controls at least the first and second light sources such that the intensity of the combined light emitted by the plurality of light sources pass below a predetermined threshold. In other words, the ionization is increased when the light sources are dimmed or switched off. This scenario is for example applicable during night-time in an office, where people are not present, but if one thoroughly wants to disinfect spaces and in particular surfaces.

According to a second aspect of the invention, there is provided a method of controlling a lighting system comprising a plurality of light sources adapted to emit light and an air ionizer adapted to generate ionized air, wherein the method comprises: individually controlling at least a first light source and a second light source of the plurality of light sources, such that at least one of color and color temperature of combined light emitted by the plurality of light sources is varied; and varying the air ionizer's generation of ionized air, either by being programmed to do so or by being controlled by a controller, in response to the controller varying at least one of the color and the color temperature of the combined light emitted by the plurality of light sources, wherein said controller is either the same controller as the controller for the light sources or a different controller. This aspect may exhibit the same or similar features and technical effect as the first aspect, and vice versa.

It is noted that the invention relates to all possible combinations of features recited in the claims.

### BRIEF DESCRIPTION OF THE DRAWINGS

This and other aspects of the present invention will now be described in more detail, with reference to the appended drawings showing embodiment(s) of the invention.

FIG. 1 is a block diagram of a lighting system according to one or more embodiments of the present invention.

FIG. 2 is a flow chart of a method to one or more embodiments of the present invention.

FIGS. 3a-d relates to ionized air generation vs. color temperature.

FIG. 4 relates to ionized air generation vs. color.

FIG. 5 shows ionized air generation vs. intensity.

In the figures, like reference numerals refer to like elements throughout.

### DETAILED DESCRIPTION

The present invention will now be described more fully hereinafter with reference to the accompanying drawings, in which currently preferred embodiments of the invention are shown. This invention may, however, be embodied in many different forms and should not be construed as limited to the embodiments set forth herein; rather, these embodiments are provided for thoroughness and completeness, and fully convey the scope of the invention to the skilled person.

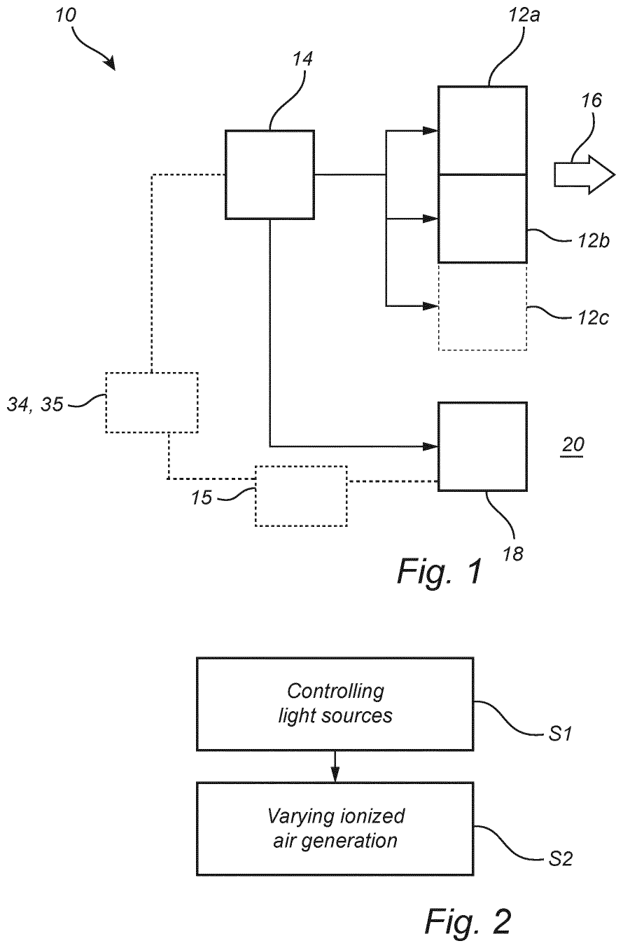

FIG. 1 is a block diagram of a lighting system 10 according to one or more embodiments of the present invention. The lighting system 10 is generally adapted to mimic natural conditions, including natural light.

The lighting system 10 comprises a plurality of light sources adapted to emit light, for example for general lighting or ambient lighting or functional lighting in a room, such as the room of an office or a home. The plurality of light sources are provided in one or more luminaires of the lighting system 10, such as a ceiling luminaire for general lighting or a wall luminaire for general lighting. Accordingly, the present lighting system 10 could be ceiling-mounted or wall-mounted.

The lighting system 10 further comprises a controller (or control unit) 14. The controller 14 is adapted to individually control at least a first light source 12a and a second light source 12b of the plurality of light sources, such that the color and/or color temperature of combined light 16 emitted by the plurality of light sources can be varied.

The first light source 12a could for example be a cool white light emitting diode (CW LED) and the second light source 12b could be a warm white (WW) LED. The color temperature of the cool white LED 12a is preferably more than 2700K, more preferably more than 3000K, most preferably more than 3300K. The color temperature of the warm white LED 12b is preferably less than 2500K, more preferably less than 2300K, most preferably less than 2200K.

Alternatively, the first light source could for example be a red light source 12a of an RGB LED and the second light source 12b could be a green light source of the RGB LED, wherein a third light source 12c of the plurality of light sources could be a(n individually controllable) blue light source of the RGB LED. The controller 14 may be connected to each of the first and second (and third) light sources 12a-b(c).

According to the present invention, the lighting system 10 further comprises an air ionizer 18. The air ionizer 18 is adapted to generate ionized air 20. The air ionizer 18 may be or comprise a (negative) ion generator. That is, the air ionizer 18 may be adapted may ionize (electrically charge) air molecules.

The air ionizer 18 is configured to vary (increase/decrease) its generation of ionized air 20 as a function of the color and/or color temperature of the combined light 16 emitted by the plurality of light sources. In other words, the air ionizer 18 may be configured to sets its ionization depending on, or in response to, the present color and/or color temperature of the emitted light 16. In this way, a lighting system 10 which better mimics natural conditions (i.e. not only natural light) can be achieved.

The air ionizer 18 may be configured to vary (increase/decrease) its generation of ionized air 20, i.e. its ionization, for example by being programmed to do so or by being controlled by a controller, which may be the same controller as the controller 14 (like in FIG. 1) or a different controller (15). Accordingly, the air ionizer 18 may be connected to the controller 14, as in FIG. 1.

The air ionizer 18 may be configured to produce a minimum ionized air concentration in the range of 25-2000 ions per cm³ and/or a maximum ionized air concentration in the range of 25000-500000 ions per cm³. The min-max may for example be about 2000-25000 ions per cm³ or about 7000-20000 ions per cm³. In this way, the lighting system 10 may protect persons from the spread of bacteria and viruses such as influenza or against the outbreak of novel viruses like COVID-19. The minimum and/or maximum ionized air concentration could for example be set by programming the air ionizer 18 or by using a sensor 35 which senses the ionized air concentration and accordingly control the amount of ionized air generated.

Upon operation of the lighting device 10, which may correspond to a method of controlling the lighting device 10, the controller 14 individually controls (at S1, see FIG. 2) at least the first light source 12a and the second light source 12b of the plurality of light sources, such that at least one of color and color temperature of the combined light 16 emitted by the plurality of light sources is varied (increased/decreased).

The controller 14, for example, also varies (at S2) the generation of ionized air of the air ionizer 18 in response to at least one of a varied color and a varied color temperature of the combined light 16 emitted by the plurality of light sources. Preferably, steps S1 and S2 (substantially) coincide in time.

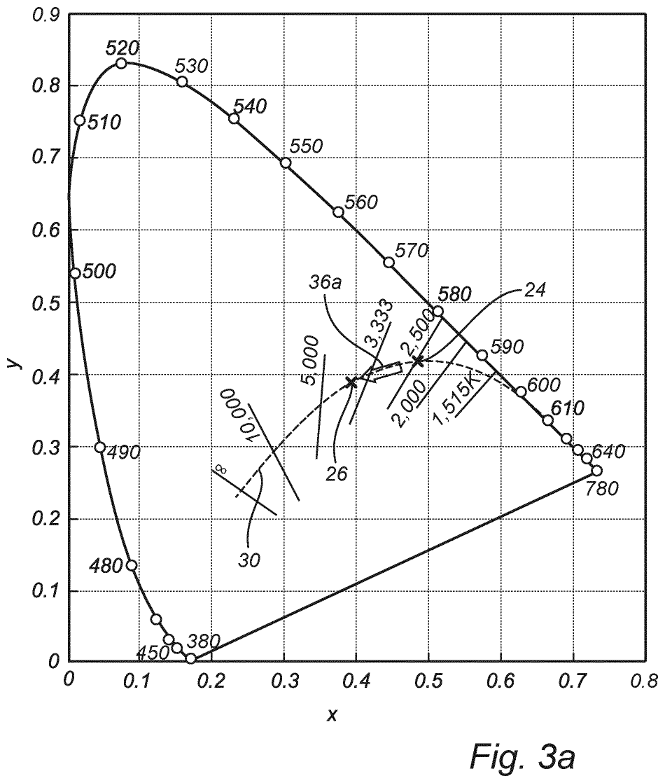

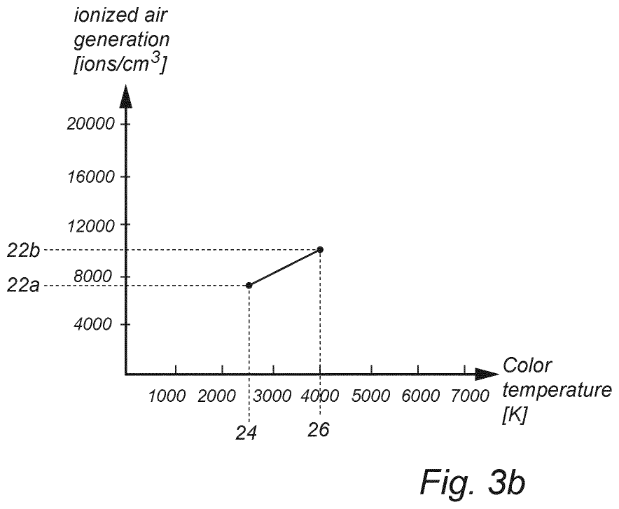

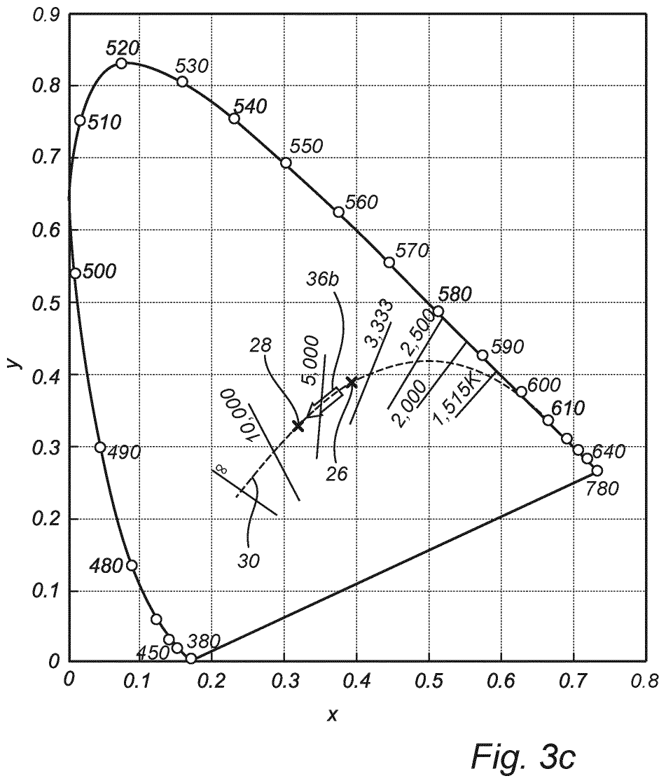

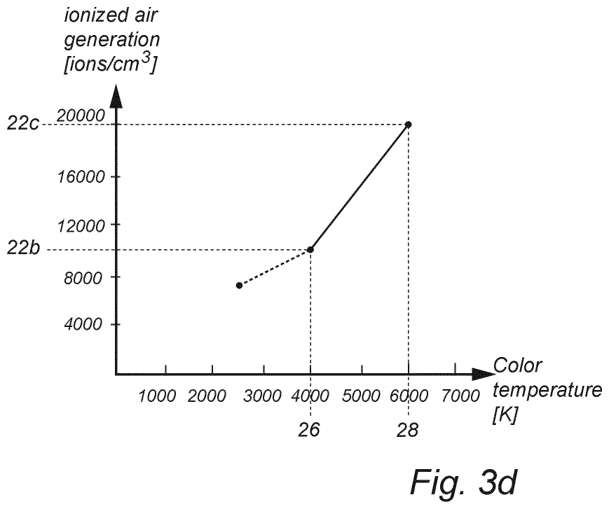

Turning to FIGS. 3a-d, the air ionizer 18 may be configured to increase its generation of ionized air 20 from a first ionized air concentration 22a to a second ionized air concentration 22b to produce a higher ionized air concentration 22b when the controller 14 controls at least the first and second light sources 12a-b such that the color temperature of the combined light 16 is increased from a first color temperature 24 to a second color temperature 26. Also, the air ionizer 18 may (likewise) be configured to decrease its generation of ionized air 20 to produce a lower ionized air concentration 22a when the controller 14 controls at least the first and second light sources 12a-b such that the color temperature of the combined light 16 is decreased from color temperature 26 to color temperature 24.

Specifically, the air ionizer 18 may be configured to increase its generation of ionized air 20 to produce the higher ionized air concentration 22b (e.g. from 7000 ions/cm³ to 11000 ions/cm³) when the controller 14 controls at least the first and second light sources 12a-b such that the color temperature of combined white light 16 is increased from the first color temperature 24, such as 2500K (which may be construed as warm white light), to the second color temperature 26, such as 4000K (which may be construed as cool white light). This increase in color temperature is illustrated by arrow 36a along the black body line 30 of the CIE 1931 color space chromaticity diagram of FIG. 3a, and the corresponding increase in ionized air generation is shown in FIG. 3b. A corresponding decrease in color temperature and ionized air generation is also envisaged.

Furthermore, the air ionizer 18 may be configured to further increase its generation of ionized air from the second ionized air concentration 22b to a third ionized air concentration 22c (e.g. from 11000 to 20000 ions/cm³) when the controller 14 controls at least the first and second light sources 12a-b such that the color temperature of the combined light 16 is further increased from the second color temperature 26 to a third, higher color temperature 28, such as 6000K (day-light). This increase in color temperature is illustrated by arrow 36b along the black body line 30 of the CIE 1931 color space chromaticity diagram of FIG. 3c, and the corresponding increase in ionized air generation is shown in FIG. 3d. A corresponding decrease in color temperature and ionized air generation is also envisaged.

As also shown in FIG. 3d, the further increase in generation of ionized air 20 when the color temperature of the combined light 16 is further increased from color temperature 26 to color temperature 28 may be steeper than the increase in generation of ionized air 20 when the color temperature of the combined light 16 sources is increased from color temperature 24 to color temperature 26. The rate between color temperatures 24 and 26 may for example be about 2.7 ions/cm³ per K, whereas the rate between color temperatures 26 and 28 may be about 4.5 ions/cm³ per K. In other words, the coefficient is greater between color temperatures 26 and 28 than between color temperatures 26 and 24. The following condition may also apply: (third color temperature 28 minus second color temperature 26)>(second color temperature 26 minus first color temperature 24) and (third ionized air concentration 22c minus second ionized air concentration 22b)>(second ionized air concentration 22b minus first ionized air concentration 22a).

Furthermore, both the increase (decrease) in generation of ionized air and the increase (decrease) in color temperature may be gradual (over time), rather than stepwise. This may serve to better mimic natural events/conditions.

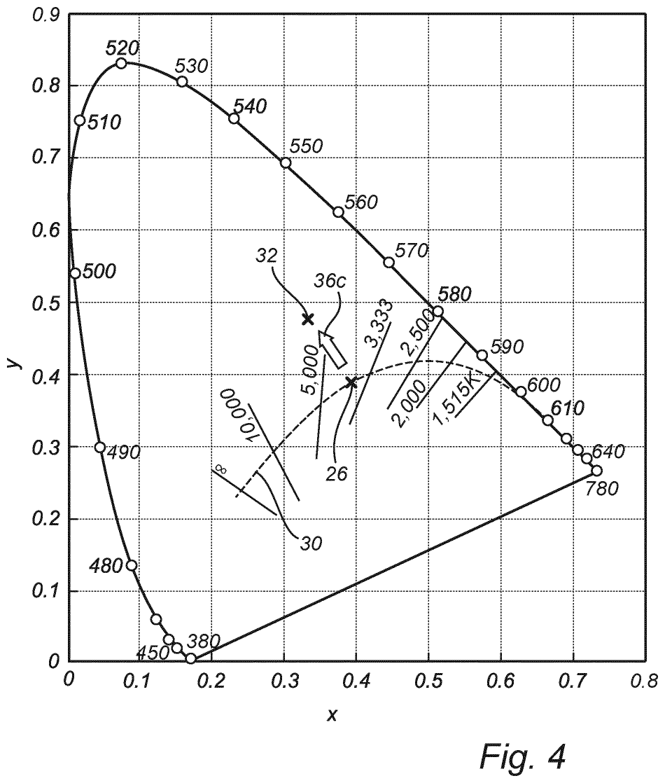

Moving on to FIG. 4, the air ionizer 18 may be configured to increase its generation of ionized air 20 to produce a higher ionized air concentration when the controller 14 controls at least the first and second light sources 12a-b such that combined white light 16 emitted by the plurality of light sources becomes more blueish and/or greenish, to produce white light with a green or blue tint. Also, the air ionizer 18 may (likewise) be configured to decrease its generation of ionized air 20 to produce a lower ionized air concentration when the controller 14 controls at least the first and second light sources 12a-b such that the combined light 16 becomes less blueish and/or greenish. Blueish white light may correspond to sky-light, and greenish white light may correspond to forest-light.

Specifically, the air ionizer 18 may be configured to further increase its generation of ionized air 20 (e.g. from the second ionized air concentration 22b to 25000 ions/cm³) when the controller 14 controls at least the first and second light sources 12a-b such that a color point of the combined light 16 moves away from the second color temperature 26 to a position 32, which may be off the black body line 30, to emit white light with a green or blue tint. The change from 26 to 32 (green tint) is illustrated by arrow 36c in the CIE 1931 color space chromaticity diagram of FIG. 4. The reverse is also envisaged.

Furthermore, the increase (decrease) in generation of ionized air 20 when the color of the combined light 16 changes may be gradual (over time), rather than stepwise. This may serve to better mimic natural events/conditions.

Furthermore, the air ionizer 18 may be configured to provide its maximum ionized air concentration (e.g. 25000 ions/cm³) when the controller 14 controls at least the first and second light sources 12a-b such that the combined light 16 is white light with a green or blue tint, like position 32 for green tint. Such light mimics the forest or waterfall, which are places where very high ionized air concentrations naturally occur.

Furthermore, the lighting system 10 may be configured such that the white light with a green or blue tint is dynamically varied as a function of time. The lighting system 10 may for example comprises means for varying at least one of the amount, position, beam shape, beam size, and pattern of (in particular green or blue light of) the emitted white light with a green or blue tint as function of time. Means for varying the amount of the emitted white light with a green or blue tint as function of time may be realized by the aforementioned controller 14. Means for varying the position and/or beam shape and/or beam size and/or pattern of the emitted white light with a green or blue tint as function of time may be realized with suitable optical means (not shown), possibly in conjunction with the controller 14 controlling such optical means. The lighting system 10 may for example project moving green light in white light, to really mimic moving leaves. Or the lighting system 10 may project moving blue light in white light, to really mimic moving a waterfall. These are typically the applications where in nature there are the highest ion concentrations. The air ionizer 18 may consequently be configured to provide its maximum ionized air concentration (e.g. 25000 ions/cm³) while the white light with a green or blue tint 16 is dynamically varied as a function of time.

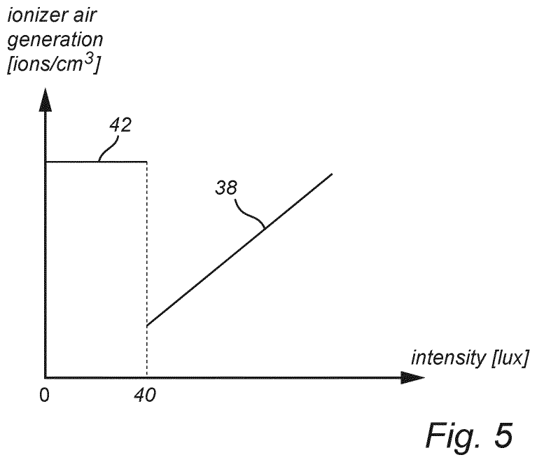

Moving on to FIG. 5, the controller 14 may further be adapted to control at least the first light source and the second light source 12a-b such that the intensity of combined light 16 can be varied, wherein the air ionizer 18 is configured to vary its generation of ionized air 20 (further) as a function the intensity of any combined light 16 emitted by the plurality of light sources.

The air ionizer 18 may for example be configured to increase (or decrease) its generation of ionized air 20 to produce a higher (or lower) ionized air concentration when the controller 14 controls at least the first and second light sources 12a-b such that the intensity of the combined light 16 is increased (or decreased), as illustrated by line 38 in FIG. 5. Furthermore, the air ionizer 18 may be configured to increase its generation of ionized air 20 to produce a higher ionized air concentration (e.g. 20000 ions/cm³) when the plurality of light sources are turned off or when the controller 14 controls at least the first and second light sources 12a-b such that the intensity of the combined light 16 pass below a predetermined threshold 40, as illustrated by line 42 in FIG. 5. The predetermined threshold 40 may for example be less than 310 lux, such as about 300 lux. In comparison, non-dimmed office lighting may be about 500 lux. This scenario is for example applicable during night-time in an office, where people are not present, but if one thoroughly wants to disinfect spaces and in particular surfaces.

Returning to FIG. 1, The lighting system may further comprise a sensor 34,35, such as a color sensor adapted to detect the color and/or color temperature of the emitted light, or a sensor adapted to sense ionized air concentration, or a presence sensor 34 adapted to detect at least one of presence and movement of one or more persons (not shown) in the vicinity of the lighting system 10. The presence sensor 34 may for example be an IR sensor. The air ionizer 18 is here configured to vary its generation of ionized air 20 based on input from the presence sensor 34. The air ionizer 18 may for example be configured to increase its generation of ionized air, or set a high generation of ionized air, in response to the presence sensor 34 detecting presence of one or more persons, and to decrease its generation of ionized air, or set a low generation of ionized air, in response to the presence sensor 34 not detecting presence of any person. The presence sensor 34 could be connected to the controller 14, in case the controller 14 controls the air ionizer 18. Alternatively, the sensor 34,35 could be connected directly to the air ionizer 18, or to some other controller (not shown) controlling the air ionizer 18.

In case the air ionizer 18 is configured to vary its generation of ionized air as a function of more than one of the color, color temperature, presence/movement, and intensity, the present lighting device 10 may have a function letting a user select what input that should take precedence. The lighting device 10 could alternatively or complementary have predetermined settings, like: during the day the presence might overrule, while during the night the light intensity might overrule.

The person skilled in the art realizes that the present invention by no means is limited to the preferred embodiments described above. On the contrary, many modifications and variations are possible within the scope of the appended claims.

Additionally, variations to the disclosed embodiments can be understood and effected by the skilled person in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

1. A lighting system, comprising:
a plurality of light sources adapted to emit light;
a control unit adapted to individually control at least a first light source and a second light source of the plurality of light sources, such that at least one of color and color temperature of combined light (16) emitted by the plurality of light sources can be varied; and
an air ionizer adapted to generate ionized air,
wherein the air ionizer is configured to vary its generation of ionized air either by being programmed to do so or by being controlled by a controller, in response to the control unit varying at least one of the color and color temperature of the combined light emitted by the plurality of light sources, wherein said controller is either the same as the control unit for the light sources or a different controller.

2. A lighting system according to claim 1, wherein the air ionizer is configured to increase its generation of ionized air from a first ionized air concentration to a second ionized air concentration to produce a higher ionized air concentration when the control unit controls at least the first and second light sources such that the color temperature of the combined light emitted by the plurality of light sources is increased from a first color temperature, such as less than 3000K, to a second color temperature, such as 3000K-4500K.

3. A lighting system according to claim 2, wherein the second ionized air concentration is at least 1.5 times the first ionized air concentration, and wherein the second color temperature minus the first color temperature is at least 500K.

4. A lighting system according to claim 2, wherein the air ionizer is configured to further increase its generation of ionized air from the second ionized air concentration to a third ionized air concentration when the control unit controls at least the first and second light sources such that the color temperature of the combined light emitted by the plurality of light sources is further increased from the second color temperature to a third color temperature,, such as greater than 4500K, wherein the third ionized air concentration is at least 1.5 times the second ionized air concentration, and wherein the third color temperature minus the second color temperature is at least 500K.

5. A lighting system according to claim 4, wherein the further increase in generation of ionized air when the color temperature of the combined light emitted by the plurality of light sources is further increased from the second color temperature to the third color temperature is steeper than the increase in generation of ionized air when the color temperature of the combined light emitted by the plurality of light sources is increased from the first color temperature to the second color temperature.

6. A lighting system according to claim 2, wherein at least one of the increase in generation of ionized air and the increase in color temperature is gradual.

7. A lighting system according to claim 1, wherein the air ionizer is configured to increase its generation of ionized air to produce a higher ionized air concentration when the control unit controls at least the first and second light sources such that combined white light emitted by the plurality of light sources becomes more blueish and/or greenish to produce white light with a green or blue tint.

8. A lighting system according to claim 7, wherein the increase in generation of ionized air is gradual.

9. A lighting system according to claim 1, wherein the air ionizer is configured to produce a minimum ionized air concentration in the range of 25-2000 ions per cm³ and/or a maximum ionized air concentration in the range of 25000-500000 ions per cm³.

10. A lighting system according to claim 1, wherein the air ionizer is configured to provide a maximum ionized air concentration when the control unit controls at least the first and second light sources such that the combined light emitted by the plurality of light sources is white light with a green or blue tint.

11. A lighting system according to claim 7, wherein the lighting system is configured such that the white light with a green or blue tint is dynamically varied as a function of time.

12. A lighting system according to claim 1, further comprising a presence sensor adapted to detect at least one of presence and movement of one or more persons in the vicinity of the lighting system, wherein the air ionizer is configured to vary its generation of ionized air based on input from the presence sensor.

13. A lighting system according to claim 1, wherein the control unit further is adapted to control at least the first light source and the second light source of the plurality of light sources such that the intensity of combined light emitted by the plurality of light sources can be varied, and wherein the air ionizer is configured to vary its generation of ionized air as a function the intensity of any combined light emitted by the plurality of light sources.

14. A lighting system according to claim 1, wherein the air ionizer is configured to increase its generation of ionized air to produce a higher ionized air concentration when the plurality of light sources are turned off or when the control unit controls at least the first and second light sources such that the intensity of the combined light emitted by the plurality of light sources pass below a predetermined threshold.

15. A method of controlling a lighting system comprising
a plurality of light sources adapted to emit light and an air ionizer adapted to generate ionized air, wherein the method comprises:
individually controlling by a control unit at least a first light source and a second light source of the plurality of light sources, such that at least one of color and color temperature of combined light emitted by the plurality of light sources is varied; and
varying the air ionizer's generation of ionized air, either by being programmed to do so or by being controlled by a controller, in response to the control unit varying at least one of the color and the color temperature of the combined light emitted by the plurality of light sources, wherein said controller is either the same as the control unit for the light sources or a different controller.
